(19) United States
(12) Patent Application Publication
(10) Pub. No.: US 2015/0060564 A1
(43) Pub. Date: Mar. 5, 2015

(54) Scent dispenser with integrated wick

(71) Applicant: Mark Bowles, Waco, TX (US)

(72) Inventor: Mark Bowles, Waco, TX (US)

(21) Appl. No.: 13/987,833

(22) Filed: Sep. 2, 2013

(51) Int. Cl. A61L 9/12

(52) U.S. Cl. CPC A61L 9/127
USPC 239/6; 239/44

(57) ABSTRACT

Apparatus and methods for dispensing scented components include a container with an opening, a closure engageable with the opening, a wick engaged with the closure and extending into the container, and a stop member engaged with the wick, the stop member having a dimension greater than the diameter of the opening for preventing removal of a portion of the wick from the container. In use, the closure can be removed from the opening to extend the wick and expose a first portion of the wick to a location. Contact between the opening and stop member retains a second portion of the wick in the container in contact with a scented component to draw the scented component toward the location.

### FIELD

Embodiments of the present disclosure relate, generally, to containers for containing and releasing scented components, and more specifically to scent dispensers usable to selectively store scented components and release scented components at a desired location.

### BACKGROUND

When hunting or performing similar activities where it is desirable to attract and/or control the location of animals, it is common to place scented lures a desired location to lure animals thereto. One type of scent dispenser can include some manner of reservoir or container having a volatile scented component therein, and some manner of porous wick or similar material that extends between and simultaneously contacts both the scented component within the container and the ambient environment external to the container. In use, the wick-like material draws the volatile scented component into the portion external to the container, where wind and/or diffusion can allow the scented component to be released into the ambient environment. As the scented component is dispersed from the external portion of the wick, the wick can continue to draw the scented component to enable prolonged use.

It is often difficult to store volatile components within a scent dispenser between uses and to transport scent dispensers between locations without losing the components, e.g., through evaporation, spillage, and/or leakage. It can also be difficult to effectively close and seal scent dispensers at times when the effectiveness of the scented components could be hindered or the components could be lost or damaged (e.g., during precipitation). Additionally, the structure of scent dispensers, being generally simple in design, is often not adapted to fully and effectively utilize the entirety of the volume of scented components within, or to efficiently distribute the weight of the dispenser and its contents when hung, suspended, or otherwise secured to an object.

A need exists for a scent dispenser that is simple in design and use, effectively supports the weight of the dispenser and its contents (e.g., at two or more points of contact), and is able to be easily and efficiently manipulated for storage, transport, and/or periods of non-use (e.g., inclement weather), and equally quickly and easily manipulated for deployment and installation.

Embodiments usable within the scope of the present disclosure meet these needs.

### SUMMARY

Embodiments usable within the scope of the present disclosure include scent dispensers and similar apparatus usable to dispense a scented compound, that include a container having at least one opening, the container being adapted for containing a scented component (e.g., a volatile liquid scent compound that evaporates/dissipates into the air and/or ambient environment when exposed thereto). For example, an embodiment can include a generally cylindrical container, having a circular base, a sidewall, and a frusto-conical upper section, terminating in an upper opening. A closure (e.g., a cap) can be removably engageable with the opening (e.g., via a threaded engagement, a force fit, a snap fit, clips/clamps or other types of fasteners, or other methods), to close the container and retain the scented component therein. In an embodiment, the closure/cap and/or the opening of the container can include a sealing material (e.g., rubber, a gasket, etc.) to seal the container and retain the scented component therein when closed.

A wick, engaged with the closure (e.g., the side thereof that faces the interior of the container), extends into the container, e.g., for contacting a scented compound inside. A stop member, engaged with the wick, can be provided with a dimension greater than the diameter of the opening of the container, to prevent removal of at least a portion of the wick. For example, in an embodiment, the stop member can include a ring through which the wick passes (e.g., the wick could be tied to the ring, adhesively secured thereto, or attached through other means), such that a first portion of the wick is positioned between the stop member and the closure, and a second portion of the wick is positioned between the stop member and the base of the container. When the closure is removed and the wick is extended outward from the container, through the opening, contact between the stop member and the interior of the container causes the second portion of the wick to be retained within the container. In an embodiment, the second portion can be provided with a length greater than or equal to the height of the container (e.g., such that the wick remains proximate to and/or in contact with the base of the container to maximize contact between the wick and the contents of the container).

Embodiments can include closures having a first side (e.g., an interior side) to which the wick is engaged, and a second side (e.g., an exterior side), having a fastener (e.g., an eye, loop, hook, etc.) extending therefrom. In use, the scent dispenser can be hung and/or suspended, such that the fastener on the closure bears a portion of the weight of the scent dispenser. Contact between the stop member and the interior of the container can cause the stop member to bear a portion of the weight of the scent dispenser as well.

In a further embodiment, the stop member can include a portion having a dimension smaller than the diameter of the opening, such that when oriented to align this portion with the opening, the stop member can be passed therethrough for enabling removal and replacement of the wick and/or the stop member.

In use, a scented component can be dispensed at a location (e.g., the ambient environment on a hunting lease) by providing a scented component into a container and transporting the container to the location. The dispenser can be deployed on an adjacent object (e.g., hung/suspended from a tree, or otherwise attached to or placed on a surface). The closure can then be removed from the opening of the container, and the wick, attached to the closure, can be extended through the opening to expose a first portion thereof to the location. In doing so, the stop member can contact the opening, such that a second portion of the wick remains in the container in contact with the scented component, such that the wick can draw the scented component toward the first portion exterior to the container to dispense the scented component at the location.

### BRIEF DESCRIPTION OF THE DRAWINGS

In the detailed description of various embodiments usable within the scope of the present disclosure, presented below, reference is made to the accompanying drawings, in which:

FIG. 1 depicts a side view of an embodiment of a scent dispenser usable within the scope of the present disclosure, in a closed position.

FIG. 2 depicts a side view of the scent dispenser of FIG. 1, in an open position.

FIG. 3 depicts a side view of an embodiment of a closure usable within the scope of the present disclosure.

FIG. 4 depicts a perspective view of an embodiment of a wick and stop member usable within the scope of the present disclosure.

FIG. 5 depicts a side view of an embodiment of a wick, closure, and stop member usable within the scope of the present disclosure.

FIG. 6A depicts a side, cross-sectional view of the scent dispenser of FIG. 1, in a closed position.

FIG. 6B depicts a side, cross-sectional view of the scent dispenser of FIG. 1, in an open position.

One or more embodiments are described below with reference to the listed Figures.

### DETAILED DESCRIPTION OF THE EMBODIMENTS

Before describing selected embodiments of the present invention in detail, it is to be understood that the present invention is not limited to the particular embodiments described herein. The disclosure and description herein is illustrative and explanatory of one or more presently preferred embodiments of the invention and variations thereof, and it will be appreciated by those skilled in the art that various changes in the design, organization, order of operation, means of operation, equipment structures and location, methodology, and use of mechanical equivalents may be made without departing from the spirit of the invention.

As well, it should be understood the drawings are intended illustrate and plainly disclose presently preferred embodiments of the invention to one of skill in the art, but are not intended to be manufacturing level drawings or renditions of final products and may include simplified conceptual views as desired for easier and quicker understanding or explanation of the invention. As well, the relative size and arrangement of the components may differ from that shown and still operate within the spirit of the invention as described throughout the present application.

Moreover, it will be understood that various directions such as “upper”, “lower”, “bottom”, “top”, “left”, “right”, and so forth are made only with respect to explanation in conjunction with the drawings, and that the components may be oriented differently, for instance, during transportation and manufacturing as well as operation. Because many varying and different embodiments may be made within the scope of the inventive concept(s) herein taught, and because many modifications may be made in the embodiments described herein, it is to be understood that the details herein are to be interpreted as illustrative and non-limiting.

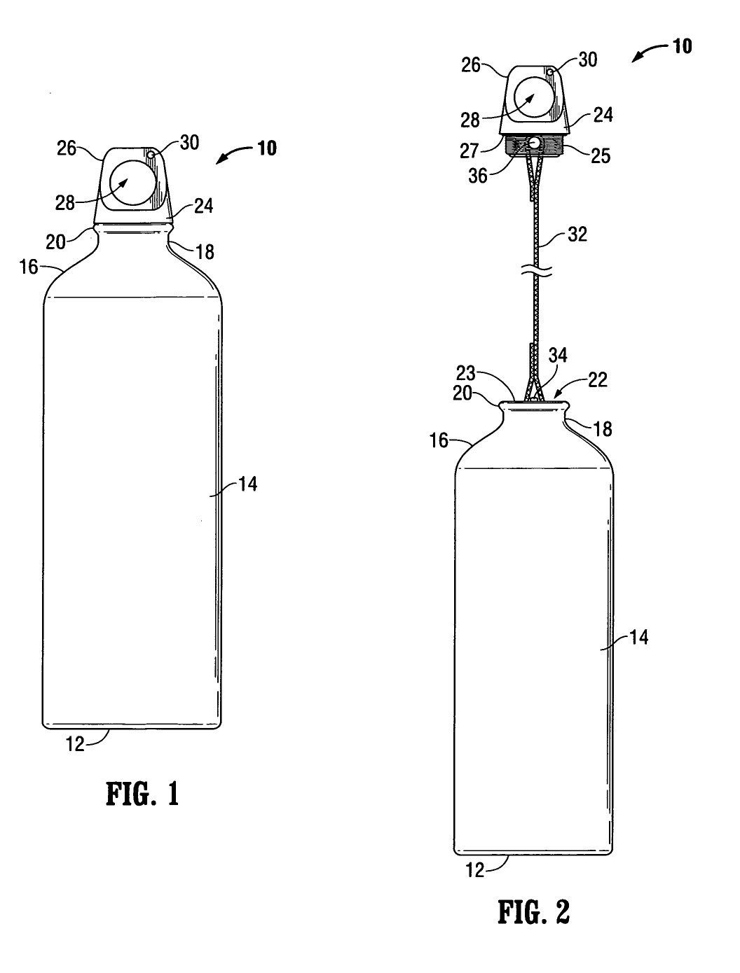

FIG. 1 depicts a side view of an embodiment of a scent dispenser (10) usable within the scope of the present disclosure. The scent dispenser (10) is shown including a container for retaining scented components (e.g., volatile liquids). While any type of container having any shape and/or dimensions can be used without departing from the scope of the present disclosure, FIG. 1 depicts the container having a generally cylindrical shape, with a circular base (12), a sidewall (14), an upper section (16) having a frusto-conical shape, and an upper cylindrical section (18) having a protruding lip (20) at the upper end thereof. Usable containers can be made from any material able to retain fluids therein, including without limitation, aluminum or other metals, plastic, wood, rubber or other polymers and/or composites, other similar materials, or combinations thereof. The exterior of the container can be treated and/or coated, e.g., to facilitate camouflage or concealment in a wilderness setting, to reflect light and/or heat to reduce evaporation of the contents, to resist wear and/or corrosion, to resist breakage, to facilitate ease of carrying, handling, and/or transport, or other similar purposes. Similarly, the interior of the container can be treated and/or coated, e.g., to minimize loss of volatile contents through the interior walls, to resist wear and/or corrosion, and/or other similar purposes.

A closure (24) (e.g., a cap) is shown engaged with the upper opening (22, shown in FIG. 2) of the container to seal and/or retain the contents therein. The depicted closure (24) is shown having a fastener (26), depicted as an eye having a central opening (28), extending from the upper side thereof. The fastener (26) is also shown including a secondary opening (30) proximate to the upper end thereof. The eye (28) and/or the secondary opening (30) are usable to receive and/or encircle objects (e.g., tree branches, cables, ropes, strings, wires, or other natural or man-made objects able to be placed at least partially through either opening (28, 30)). It should be understood what while FIG. 1 depicts a closure (24) having an eye-type fastener, other types of fasteners, such as hooks, flexible objects (e.g., wires, cords, ropes, cables, etc.), hook and loop type fasteners, adhesive-type fasteners, or any other type of fastening mechanism could be used without departing from the scope of the present disclosure. Further, in other embodiments, use of a fastener could be omitted, and the scent dispenser (10) could be secured using other means (e.g., a separate fastener external to the scent dispenser (10), looping and/or tying the wick (32, shown in FIG. 2) about an object, placing the container on top of a supporting surface, etc.)

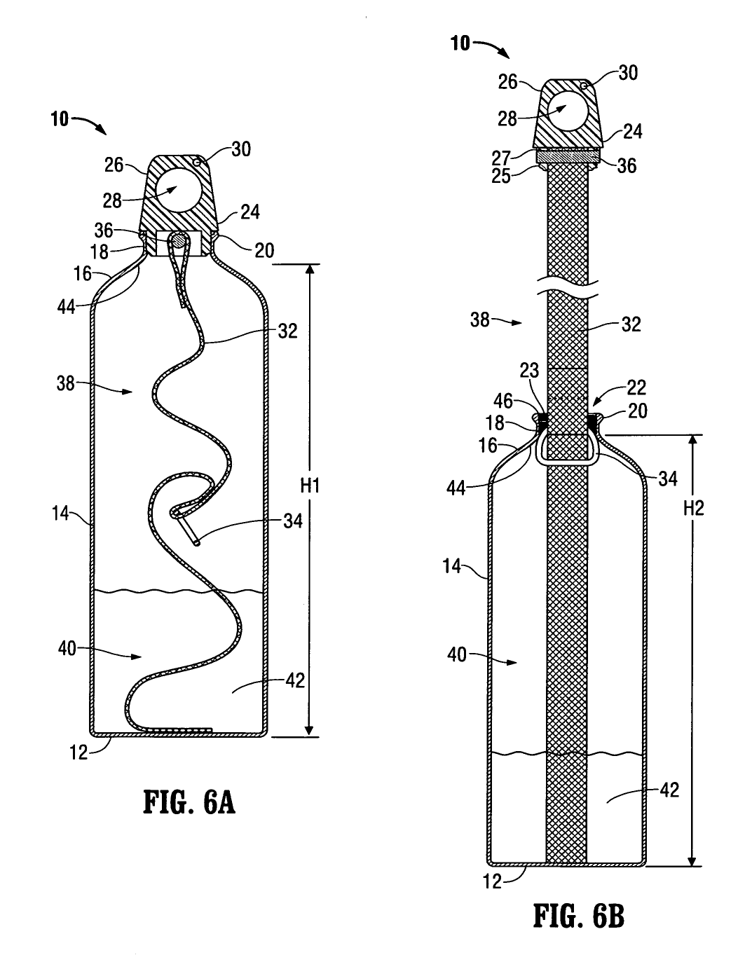

FIG. 2 depicts the scent dispenser (10) of FIG. 1 in an open position, such that the container is depicted hanging and/or suspended from the closure (24), which can be secured to an object using the fastener (26). As described above, the container is shown including a base (12), a sidewall (14), an upper section (16), an upper cylindrical section (18), and a lip (20), defining an opening (22) in the top of the container. The opening (22) is shown having internal threads (23, better shown in FIG. 6B), which are engageable with external threads (25) associated with the closure (24). In other embodiments, the closure (24) could be provided with internal threads while the container is provided with external threads. In still other embodiments, the container could be engaged with the closure (24) through other means, such as a snap-fit, force-fit, compression-fit, clips, clamps, clasps, or other types of fasteners. The closure (24) is also shown including a sealing material (27) (e.g., rubber, plastic, a gasket-type material, etc.) such that when secured to the container, the sealing material (27) forms a seal against the lip (20) such that the contents of the container are prevented from spilling and/or otherwise escaping through the opening (22) (e.g., via evaporation). While FIG. 2 depicts a sealing material (27) disposed on the closure (24), it should be understood that embodiments could include sealing material disposed on the container, the closure (24), the internal or external threads (23, 25), or combinations thereof, or in other embodiments, use of a sealing material could be omitted.

A wick (32) is shown engaged with the closure (24) (e.g., to a pin (36) extending therethrough) and extending into the container through the opening (22). While any type of material capable of conveying a fluid from the interior of the container through the wick (32) can be used, FIG. 2 depicts a wick (32) similar to that used with a kerosene lantern or similar type of device, able to draw fluid upward along the wick material and through the spaces therein. The wick (32) can have any desired thickness, width, and length, depending on the surface area desired for exposure to the ambient environment external to the container. For example, a wick having a larger surface area could dispense a larger quantity of a scented component into the air surrounding the dispenser (10). While FIG. 2 depicts a portion of the wick (32) external to the container and extending between the closure (24) and the container opening (22), a second portion of the wick (not visible in FIG. 2) can remain within the container in contact with the contents thereof. A stop member (34) is shown associated with an intermediate point along the length of the wick to prevent withdrawal of the entire wick (32) from within the container.

While any type of stop member having at least one dimension larger than the diameter of the opening (22) can be used without departing from the scope of the present disclosure, FIG. 2 depicts the stop member (34) as a D-shaped ring member (better shown in FIG. 4), having a length greater than that of the diameter of the opening (22) and a width slightly less than the diameter of the opening (22). As such, contact between the stop member (34) and the interior of the container can prevent inadvertent removal of the entirety of the wick (32) from the container, but when desired, the stop member (34) can be manipulated to position the narrower width thereof into alignment with the opening (22) to enable removal of the wick (32) and stop member (34) from the container, e.g., for cleaning, replacement, repair, etc.

The depicted configuration of components shown in FIG. 2 enables a portion of the weight of the scent dispenser (10) and the contents therein to be supported by the fastener (26) and closure (24), and a portion of the weight to be supported by stop member (34) and wick (32).

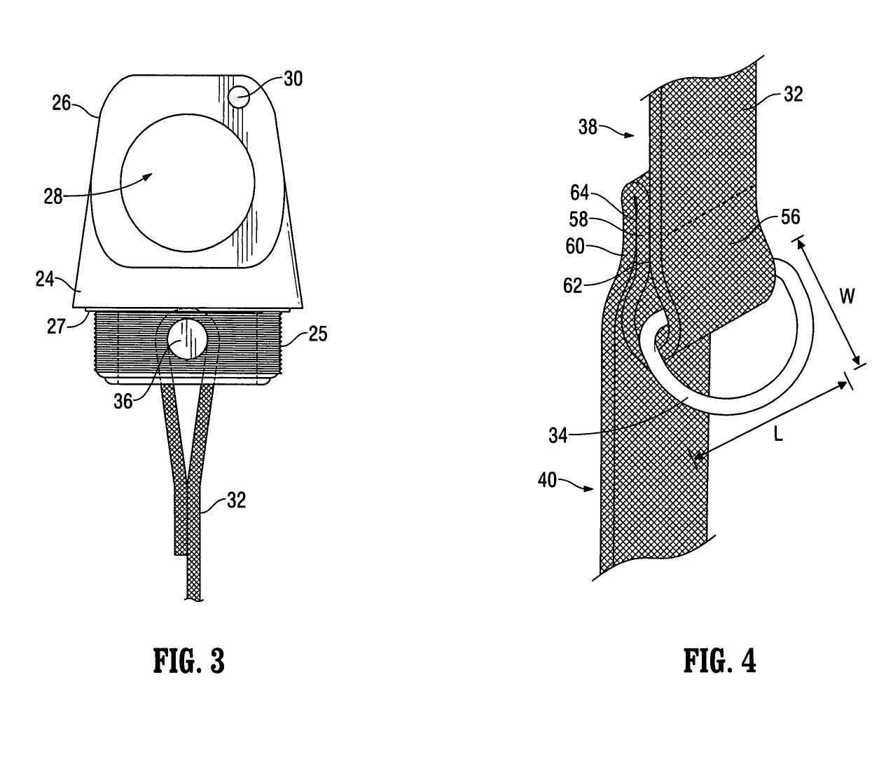

FIG. 3 depicts a side view of an embodiment of the closure (24), usable within the scope of the present disclosure. As described above, the depicted closure (24) includes a fastener (26) having a central opening (28) and a secondary opening (30) extending from the upper surface thereof, and a sealing material (27) and external threads (25) associated with the lower surface thereof. In the depicted embodiment, a pin (36) extending through the portion of the closure (24) having the external threads (25), is used to secure the wick (32) to the closure, e.g., by looping the wick (32) around the pin (36) and adhesively securing two portions of the wick (32) to one another. It should be understood that the depicted method of securing the wick (32) to the closure (24) is an exemplary embodiment, and that a pin (36) could be . positioned at any point in the closure (24), the wick (32) could be tied, sewn, snapped, clipped, clamped, and/or otherwise secured to the pin (36), or alternatively, use of a pin (36) could be omitted, and the wick (32) could be directly secured to any portion of the closure (24) through other means (e.g., adhesives, fasteners, etc.)

FIG. 4 depicts a perspective view of an embodiment of the wick (32) and stop member (34), usable within the scope of the present disclosure. As described above, the wick (32) is shown having a D-shaped, ring-type stop member (34) secured at an intermediate point along the length thereof (e.g., by looping the wick through the stop member (34) and adhesively securing two portions of the wick (32) to one another), thereby conceptually dividing the wick (32) into a first portion (38) on one side of the stop member (34), and a second portion (40) on the opposite side of the stop member (34). The depicted stop member (34) is shown having a length (L) longer than a width (W) thereof. The dimensions of the stop member (34) can be selected such that one dimension (e.g., the length (L)) is greater than the diameter of the opening (22, shown in FIG. 2) of the scent dispenser container, while another dimension (e.g., the width (W)) is less than the diameter of the opening. As such, during use, contact between the stop member (34) and the interior of the container can prevent unintentional removal of the entirety of the wick (32) and stop member (34) therefrom, due to the length (L) of the stop member (34) causing such contact; however, manipulation of the stop member (34) to align the width (W) with the opening can allow the stop member (34) to pass therethrough and enable removal of the wick (32) and stop member (34).

It should be understood that the depicted stop member (34) is a single exemplary embodiment, and that any member having at least one dimension larger than the diameter of the opening of the container can be used without departing from the scope of the present disclosure. For example, in an embodiment, a stop member (34) that is unable to pass through the opening could be used (e.g., a stop member (34) lacking any dimension smaller than the diameter of the opening). In other embodiments, a larger or smaller stop member (34) could be used, e.g., to vary the point along the interior of the container that will be contacted by the stop member (34) to control the length of wick (32) that is extendable from the container. Additionally, while FIG. 4 depicts the wick (32) looped through a ring-type stop member (34) and adhesively secured (e.g., through engagement of folds (56, 58, 60) of the wick (32) using adhesives (62, 64)), it should be understood that in other embodiments, the wick (32) could be secured via sewing, clips, snaps, clamps, and/or other types of fasteners, and depending on the shape of the stop member (34), the wick could be tied thereto, wrapped about the stop member (34), the stop member (34) could penetrate or otherwise pass through an orifice in the wick (32), the wick (32) could pass through a slot or other type of orifice in the stop member (34), or other methods of securing could be used.

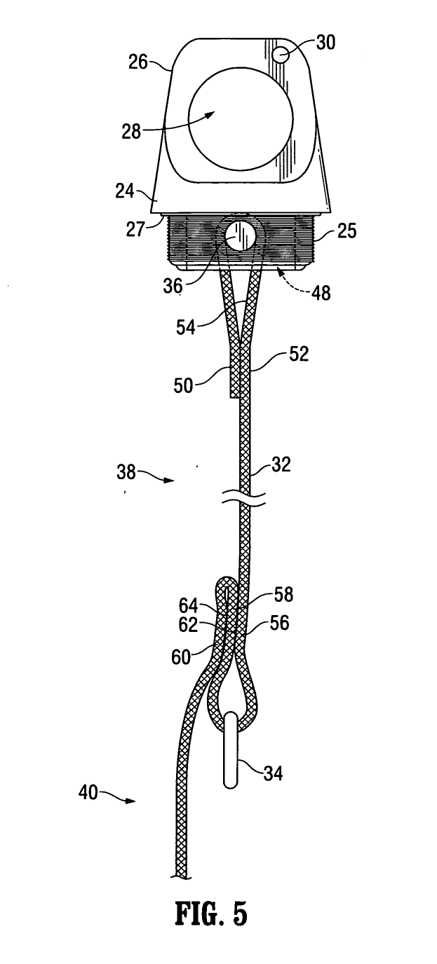

FIG. 5 depicts a side view of an embodiment of the closure (24), wick (32), and stop member (34) usable within the scope of the present disclosure. As described previously, the closure (24) is shown having a fastener (26) with a central opening (28) and a secondary opening (30) extending from an upper surface thereof, and sealing material (27) and external threads (25) associated with a lower surface thereof. The pin (36) is shown extending through the portion of the closure (24) that includes the external threads (25), but as described previously, other means to secure the wick (32) to the closure (24) could also be used. The lower portion of the closure (24) that includes the external threads (25) is shown having a generally hollow, cylindrical shape with a lower opening (48) through which the wick (32) extends to engage the pin (36). Specifically, the end portion of the wick (32) is shown looped over and/or around the pin (36), such that a first overlapping region (50) proximate to the end of the wick (32) is positioned adjacent to a second overlapping region (52). The overlapping regions (50, 52) are shown secured to one another using adhesive (54), though as described previously, other means of securing the overlapping regions (50, 52) (e.g., sewing, hook and loop fasteners, clips, clamps, etc.) could be used without departing from the scope of the present disclosure. Alternatively or additionally, the wick (32) could be tied about the pin (36) engaged to and/or through an orifice in the pin (36) or other portion of the closure (24), or secured using other similar methods.

The stop member (34) is shown engaged with an intermediate point along the length of the wick (32), thereby dividing the wick (32) into the first portion (38) and second portion (40), as described above. FIG. 5 depicts the wick (32) secured to the stop member (34) by passing and/or looping the wick (32) through the stop member (34), such that a third overlapping region (56) is positioned adjacent to a fourth overlapping region (58). In the depicted embodiment, the wick (32) is folded proximate to the fourth overlapping region (58) to position a fifth overlapping region (60) adjacent thereto. Adhesives (62, 64) are shown securing the overlapping regions (56, 58, 60) to one another, though as described above, other means of securing a wick (32) to a stop member (34) can also be used.

FIGS. 6A and 6B depict side, cross-sectional views of the scent dispenser (10) of

FIG. 1, in closed and open positions, respectively. FIG. 6A shows the dispenser (10) including a container having the base (12), sidewall (14), the upper section (16) having a frusto-conical shape, the upper cylindrical opening (18), and the lip (20). The container is shown having a volume of a scented component (42) within, proximate to the base (12) and contained using the base (12) and sidewall (14). The closure (24) is shown engaged with the container to close and seal the opening (22, shown in FIG. 6B), using complementary internal and external threads (23, 25, shown in FIG. 6B) and the sealing material (27, shown in FIG. 6B). The closure (24) includes a fastener (26) having central and secondary openings (28, 30), as described previously.

The wick (32) is shown engaged with the closure (24) via the pin (36), such that the wick (32) extends downward from the closure (24), through the interior of the container, and into contact with the volume of scented component (42) proximate to the base (12). The stop member (34) is shown engaged with an intermediate point along the length of the wick (32), thereby dividing the wick into a first portion (38) extending between the closure (24) and the stop member (34), and a second portion (40) extending between the stop member (34) and the base (12) of the container. The interior of the container, proximate to the opening functions as a contact surface (44), adapted to contact the stop member (34) when the wick (32) is at least partially extracted from the container.

While the scent dispenser (10) is in the closed position, as depicted in FIG. 6A, the scented component (42) within is prevented from spillage and/or loss via evaporation due to the closure (24) and associated seal, e.g., between the sealing material (27) and the upper surface (46) of the container, thereby facilitating transport and/or storage of the scent dispenser (10), as well as protection and containment of the components therein during periods of non-use.

FIG. 6B depicts the scent dispenser (10) in an open, deployed position.

Specifically, the closure (24) is shown removed from the opening (22), and displaced outward therefrom to extend the first portion (38) of the wick (32) through the opening (22) and external to the container. Contact between the stop member (34) and the contact surface (44) within the container prevents further extraction of the wick (32) through the opening (22), such that the second portion (40) of the wick (32) is retained within the container.

The container is depicted having a height (H1), which, in combination with the shape and other dimensions of the container, can determine the maximum volume able to be contained therein. FIG. 6B depicts the second portion (40) of the wick (32) having a height (H2) generally equal to that of the height (H1) of the container, such that the lower end of the wick (32) remains in contact with and/or generally proximate to the base (12) of the container.

In use, after removal of the closure (24) from the opening (22) and extraction of the first portion (38) of the wick (32) from the container, the fastener (26) can be used to engage and/or secure the scent dispenser (10) to a desired object (e.g., a limb of a tree or a similar type of natural or man-made object having at least one dimension capable of passing through the central or secondary openings (28, 30)). The fastener (26) thereby supports a first portion of the weight of the container and scented component (42) therein, while contact between the stop member (34) and the contact surface (44) within the container supports a second portion of the weight. The height (H2) of the second portion of the wick (32), relative to the height (H1) of the container, serves to retain the wick (32) in contact with the scented component (42), independent of the remaining volume thereof. The scented component (42) is thereby drawn toward the opening (22) by the wick (32), passing through the second portion (40), through the opening (22) to the first portion (38), where it is exposed to the environment external to the container. As scent from the first portion (38) of the wick (32) is dispersed into the environment (e.g., due to wind, moisture, and/or evaporation), the wick (32) can continue to draw the scented component (42) from within the container toward the first portion (38) to continuously provide a scent into the ambient environment.

Embodiments described herein thereby provide scent dispensers and methods in which the weight of the dispenser and its contents can be favorably supported (e.g., using two or more points of contact), that can be easily and efficiently manipulated (e.g., opened and closed) for deployment and installation, storage, transport, and/or periods of non-use.

While various embodiments usable within the scope of the present disclosure have been described with emphasis, it should be understood that within the scope of the appended claims, the present invention can be practiced other than as specifically described herein.

What is claimed is:

1. An apparatus for dispensing a scented component, the apparatus comprising:
a container having an opening, wherein the container is adapted for containing the scented component;
a closure removably engageable with the opening for closing the container to retain the scented component therein;
a wick engaged with the closure and extending into the container; and
a stop member engaged with the wick, wherein the stop member comprises a dimension greater than a diameter of the opening for preventing removal of a portion of the wick from the container.

2. The apparatus of claim 1, wherein the container comprises a cylindrical shape having a generally circular base, a sidewall, and a generally frustoconical upper section.

3. The apparatus of claim 1, wherein the closure is threadably engageable with the opening of the container.

4. The apparatus of claim 1, wherein the closure, the opening of the container, or combinations thereof comprise a compressible sealing material for sealing the container to retain the scented component therein.

5. The apparatus of claim 1, wherein the closure comprises a first side having the wick engaged therewith and a second side having a fastener extending therefrom, wherein the fastener is adapted to secure the container to an adjacent object.

6. The apparatus of claim 1, wherein the wick comprises a first portion disposed between the stop member and the closure and a second portion disposed between the stop member and an interior surface of the container.

7. The apparatus of claim 6, wherein the second portion of the wick comprises a length greater than or equal to a height of the container.

8. The apparatus of claim 1, wherein the stop member comprises a ring and wherein the wick passes through the ring.

9. The apparatus of claim 1, wherein the stop member comprises a portion having an additional dimension smaller than the diameter of the opening for enabling removal and replacement of the wick, the stop member, or combinations thereof.

10. A method for dispensing a scented component to a location, the method comprising the steps of:
providing a container containing the scented component to the location;
removing a closure from an opening of the container and extending a wick engaged with the closure through the opening to expose a first portion the wick to the location; and
contacting the opening with a stop member engaged with the wick and comprising a dimension greater than a diameter of the opening, such that a second portion of the wick is retained in the container in contact with the scented component, wherein the wick draws the scented component toward the first portion to dispense the scented component to the location.

11. The method of claim 10, wherein the step of removing the closure from the opening of the container comprises unthreading the closure from the opening.

12. The method of claim 10, further comprising the step of inserting the first portion of the wick through the opening and into the container, engaging the closure with the opening to close the container, and removing the container from the location.

13. The method of claim 10, wherein the step of providing the container to the location comprises engaging a fastener extending from the closure to an object.

14. The method of claim 13, wherein the step of contacting the opening with the stop member comprises supporting a weight of the container containing the scented component using the fastener and the stop member.

15. The method of claim 10, wherein the step of contacting the opening with the stop member such that the second portion of the wick is retained in the container in contact with the scented component comprises retaining the second portion of the wick in contact with a base of the container.

16. The method of claim 10, further comprising the step of removing the wick and the stop member from the container for replacement by passing a portion of the stop member having an additional dimension smaller than the diameter of the opening through the opening.

17. A scent dispenser comprising:
a container having a base, a sidewall, an opening, and an interior;
a cap engageable with the opening, wherein the cap comprises an outer surface and an inner surface;
a fastener on the outer surface of the cap, wherein the fastener is adapted to engage the cap and the container to an adjacent object;
a wick engaged with the inner surface of the cap and extending into the interior of the container; and
a stop member engaged with the wick, wherein the wick comprises a first portion positioned between the stop member and the cap and a second portion positioned between the stop member and the base, wherein the stop member comprises a dimension greater than a diameter of the opening for preventing removal of the second portion portion of the wick from the container, wherein the second portion of the wick comprises a length greater than or equal to a height of the container, and wherein the fastener and the stop member are adapted to support a weight of the container.

18. The scent dispenser of claim 17, wherein the stop member comprises a ring and wherein the wick passes through the ring.

19. The scent dispenser of claim 17, wherein the stop member comprises a portion having an additional dimension smaller than the diameter of the opening for enabling removal and replacement of the wick, the stop member, or combinations thereof.

20. The scent dispenser of claim 17, wherein the inner surface of the cap comprises a pin extending therethrough, and wherein the wick is engaged with the pin.
